(19) United States
(12) Patent Application Publication
(10) Pub. No.: US 2012/0203313 A1
(43) Pub. Date: Aug. 9, 2012

(54) DETACHABLE HEAT-RETAINING GLOVES

(76) Inventor: Jong Sook Kim, Seoul (KR)

(21) Appl. No.: 13/502,053

(22) PCT Filed: Oct. 11, 2010
(86) PCT No.: PCT/KR2010/006919

§ 371 Date: Apr. 13, 2012

(30) Foreign Application Priority Data
Oct. 21, 2009 (KR) 10-2009-0100006
Mar. 4, 2010 (KR) 10-2010-0019349

(51) Int. Cl. A61F 7/00

(52) U.S. Cl. 607/111

(57) ABSTRACT

Detachable heat-retaining gloves apply to baby carriages, silver cars, wheelchairs, hand cars, pet carriages, hang gliders, bicycles, sticks, and so on in order to keep hands warm and make them cold resistance in the winter season, which is detachably mounted on any baby carriage, which allows a user to freely moves his or her hands, and which can prevent a loss of the gloves through the user's carelessness since there is no need to separately keep the gloves after taking off them. Each of the detachable heat-retaining gloves includes a body of a tumbling doll shape symmetrically formed and being foldable at a central portion thereof; a heat-retaining member joined along the inner face of the body; an outer skin member joined along the outer face of the body; first and second zippers joined along both side edges of the body to form a receiving portion inside the body; and a separation-preventing ring mounted at one side of the inner face of the body, wherein the body further comprises a cross-shaped (+) or straight-shaped (−) fitting portion formed on one side of a lower portion of the body.

### BACKGROUND OF THE INVENTION

The present invention relates to detachable heat-retaining gloves, and more particularly, to detachable heat-retaining gloves, which can be applied to baby carriages, silver cars, wheelchairs, hand cars, pet carriages, hang gliders, bicycles, sticks, and so on in order to keep hands warm and make them cold resistance in the winter season, which is detachably mounted on any baby carriage, which allows a user to freely moves his or her hands, and which can prevent a loss of the gloves through the user's carelessness since there is no need to separately keep the gloves after taking off them.

In general, gloves are used for protecting hands when a user works or drives with hands or uses a baby carriage or used for ornamental purposes, but are the most used for protecting hands from the outside cold air and keeping them warm in the winter season.

As described above, each of the cold-resistant gloves includes an outer skin made of leather, vinyl, artificial leather, waterproof fabric, synthetic fiber, and so on and an inner skin mainly made of natural fiber or synthetic fiber. The outer skin is put on top of the inner skin and they are sewed together to thereby form gloves.

Here, the outer skin made of one of leather, vinyl, artificial leather, and others, which are materials tough and airtight, is used for shutting off the outdoor air, and the inner skin made of natural fiber or synthetic fiber is used for enhancing feeling of where a hand touches.

However, if the outer skin gets thicker in order to more effectively shut off the outdoor cold air, it causes inconvenience in freely moving the hands in the gloves.

Furthermore, if the gloves are made of cotton-like fabric or has a thin outer skin in order to allow a free movement of the hands, it causes deterioration of heat-retaining function due to introduction of the outdoor cold air.

In order to solve the above-mentioned problems, Korean Patent Application No. 20-2008-0009017 discloses heat-retaining arm warmers for motorcycles.

The heat-retaining arm warmers for motorcycles according to the prior art includes: a heat-retaining part having an outer skin and an inner skin; a hand hole which is an entrance of the heat-retaining part; and an electric heater having an insulating sheet, a heat-generating resistor arranged on the insulating sheet along a predetermined pattern and a switch for making and breaking the heat-generating resistor, the electric heater having heating wires embedded therein to thereby keep the inside warm within a necessary temperature range.

However, the heat-retaining arm warmers according to the prior art has several problems in that it is inconvenient to attach and detach and in that it is only used for a special purpose, such as driving of motorcycles due to its thick outer skin and provides a bad appearance when it is applied to other articles.

### SUMMARY OF THE INVENTION

Accordingly, the present invention has been made to solve the above-mentioned problems occurring in the prior arts, and it is an object of the present invention to provide detachable heat-retaining gloves, each of which includes a body of a tumbling doll shape being foldable at a central portion thereof, zippers joined to the body along both side edges of the body to thereby form a receiving portion, such that it can protect a user's hands from the outdoor cold air in the winter season and allow the user to freely move the hands.

It is another object of the present invention to provide detachable heat-retaining gloves, each of which includes a cross-shaped or straight-shaped fitting portion formed on a lower portion of the body, thereby being applicable to various kinds of baby carriages, silver cars, wheelchairs, hand cars, pet carriages, hang gliders, bicycles, sticks, and so on.

It is a further object of the present invention to provide detachable heat-retaining gloves, which further includes a Velcro fastener mounted at an end portion of the fitting portion, thereby preventing wind blowing into the receiving portion.

To accomplish the above object, according to the present invention, there is provided heat-retaining gloves, each of which includes: a body of a tumbling doll shape symmetrically formed and being foldable at a central portion thereof; a heat-retaining member joined along the inner face of the body; an outer skin member joined along the outer face of the body; first and second zippers joined along both side edges of the body to form a receiving portion inside the body; and a separation-preventing ring mounted at one side of the inner face of the body, wherein the body further comprises a cross-shaped (+) or straight-shaped (−) fitting portion formed on one side of a lower portion of the body.

Furthermore, buttons, Velcro tapes or magnets are capable of being mounted on the body in place of the zippers.

Moreover, it is preferable that the present invention may further include heating wires interposed between the heat-retaining member and the outer skin member.

Additionally, it is preferable that the present invention may further include wrist tightening portions extending from an upper end portion and a lower end portion of the body and formed symmetrically to form an entrance of the receiving portion.

In addition, it is preferable that the present invention may further include a Velcro fastener mounted at an end portion of the fitting portion in order to prevent wind blowing into the receiving portion.

As described above, the detachable heat-retaining gloves according to the present invention can keep the user's hands warm and prevent the hands from the cold air in the winter season. Since they can be mounted on one of various kinds of baby carriages or the likes.

Furthermore, since a hand-grip of the baby carriage is located inside the receiving portion of the heat-retaining glove, the user can freely move the hand inside the receiving portion.

Moreover, since the body of the glove includes the zippers joined to both side edges thereof in an openable manner and the cross-shaped or straight-shaped fitting portion formed at the lower portion thereof, the detachable heat-retaining gloves according to the present invention can be detachably mounted and applied to one of baby carriages, silver cars, wheelchairs, hand cars, pet carriages, hang gliders, bicycles and sticks.

Additionally, the detachable heat-retaining gloves according to the present invention can enhance the warm-keeping effect and the cold-resistant effect since the Velcro fastener is disposed at the end portion of the fitting portion to prevent wind blowing into the receiving portion.

In addition, since the gloves are detachably mounted on any baby carriage and each has the separation-preventing ring, the detachable heat-retaining gloves according to the present invention can prevent a loss of the gloves through the user's carelessness as there is no need to separately keep the gloves after taking off them.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 illustrates detachable heat-retaining gloves according to the present invention;

FIGS. 2a to 2c illustrate a spread state of the heat-retaining gloves;

FIG. 3 illustrates the detachable heat-retaining gloves according to a first preferred embodiment of the present invention;

FIG. 4 illustrates the detachable heat-retaining gloves according to a second preferred embodiment of the present invention; and

FIGS. 5 to 7 illustrate a used state of the detachable heat-retaining gloves according to the present invention.

### DETAILED DESCRIPTION OF THE PREFERRED EMBODIMENT

Now, detachable heat-retaining gloves according to preferred embodiments of the present invention will be described. In the description of the present invention, when it is judged that detailed descriptions of known functions or structures related with the present invention may make the essential points vague, the detailed descriptions of the known functions or structures will be omitted.

Moreover, the terms in the following detailed description of the present invention are terms defined in consideration of functions in the present invention and may be changed according to users' purposes or customs.

Hereinafter, reference will be now made in detail to the preferred embodiment of the present invention with reference to the attached drawings.

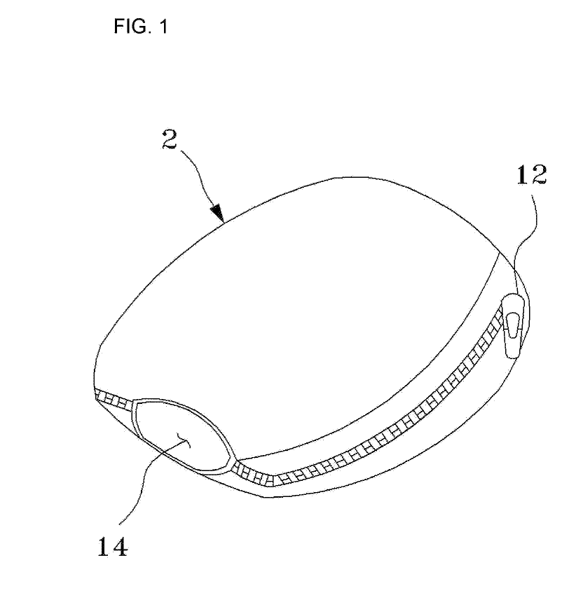

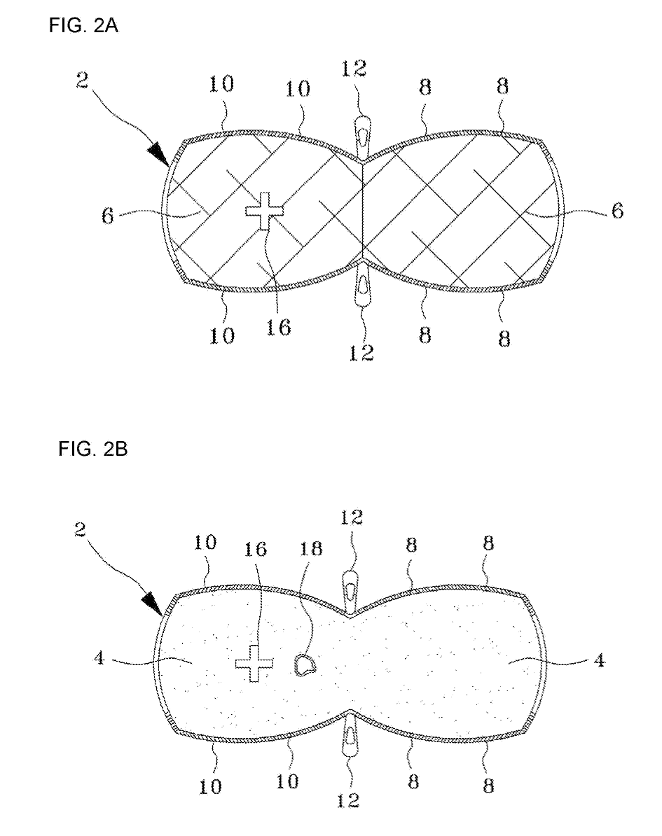

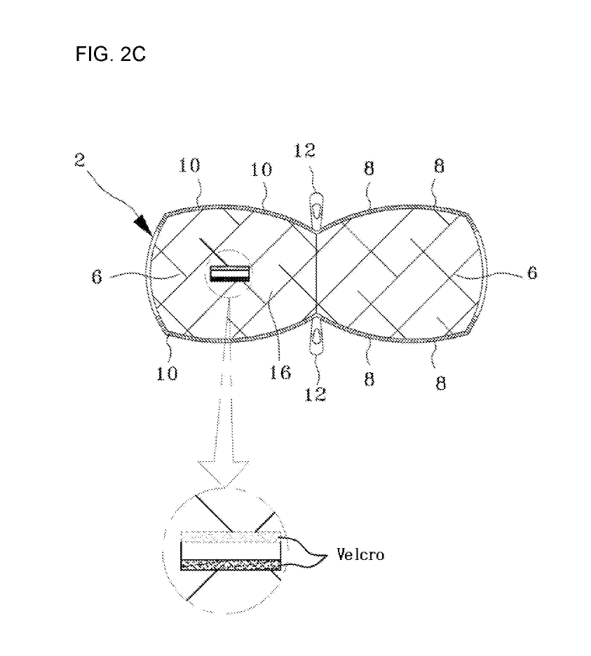

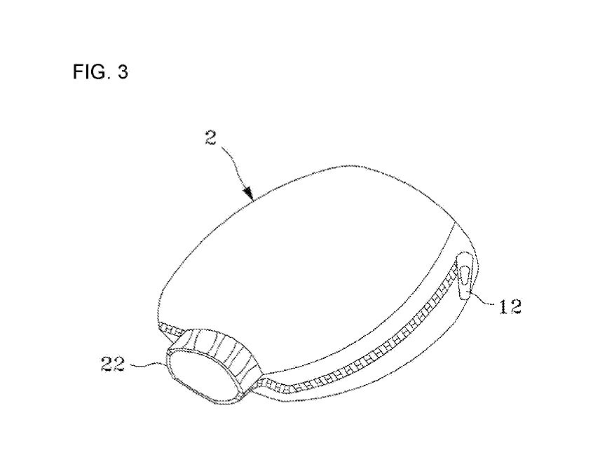

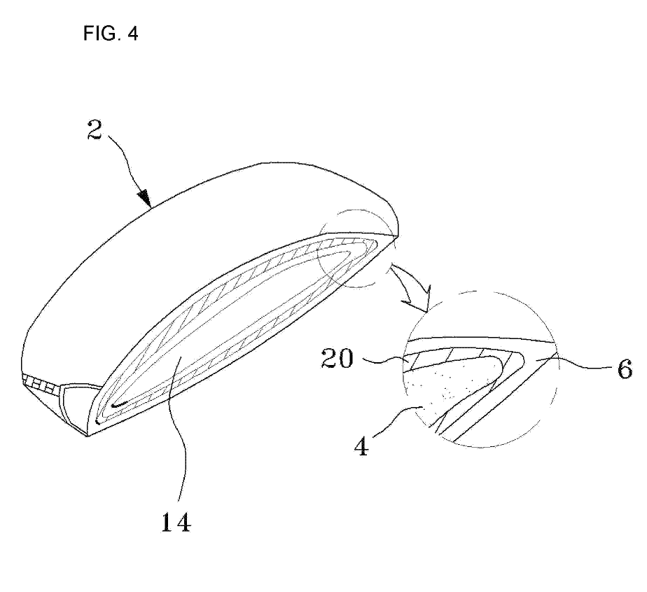

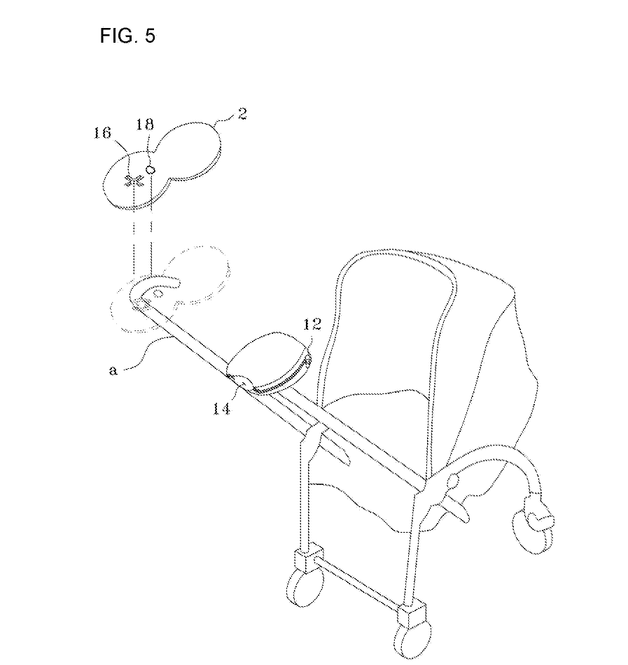

FIG. 1 illustrates detachable heat-retaining gloves according to the present invention, FIGS. 2a to 2c illustrate a spread state of the heat-retaining gloves, FIG. 3 illustrates the detachable heat-retaining gloves according to a first preferred embodiment of the present invention, FIG. 4 illustrates the detachable heat-retaining gloves according to a second preferred embodiment of the present invention, and FIGS. 5 to 7 illustrate a used state of the detachable heat-retaining gloves according to the present invention.

Referring to the drawings, each of the detachable heat-retaining gloves includes: a body 2 of a tumbling doll shape symmetrically formed and being foldable at the center thereof; a heat-retaining member 4 disposed along the inner face of the body 2; and an outer skin member 6 disposed along the outer face of the body 2.

Here, it is preferable that the heat-retaining member 4 is mainly made of natural fiber or synthetic fiber and the outer skin member 6 is made of leather, vinyl or artificial leather.

Furthermore, a first zipper and a second zipper are symmetrically joined along both edges of the body 2, such that a receiving portion 14 is formed inside the body 2. In this instance, each of the first and second zippers includes: a male zipper 8 formed along both side edges of an upper portion of the body 2; a female zipper 10 formed along both side edges of a lower portion of the body 2; and a zipper slide 12 interposed between the male zipper 8 and the female zipper 10 to engage the male zipper 8 and the female zipper 10 with each other.

Here, in place of the male zipper 8, the female zipper 10 and the zipper slide 12, buttons, Velcro tapes or magnets may be used.

Moreover, the receiving portion 14 is formed when the male zippers 8 and the female zippers 10 are engaged with each other by the zipper slides 12.

Additionally, the body 2 further includes a fitting portion 16 penetratingly formed on a side of the bottom thereof. In this instance, the fitting portion 16 is generally formed in a cross shape as shown in FIG. 2b, but may be formed in a straight shape as shown in FIG. 2c.

The body 2 may further include a Velcro fastener mounted at an end portion of the fitting portion 16 in order to prevent wind blowing into the receiving portion.

Furthermore, the body 2 may further include a separation-preventing ring 18 formed on a side of the inner face thereof and a heating wire 20 arranged between the heat-retaining member 4 and the outer skin member 6.

In addition, the body 2 may further include wrist tightening portions 22 extending from an upper end portion and a lower end portion of the body 22 and formed symmetrically to form an entrance of the receiving portion 14 formed when the male zipper and the female zipper are engaged with each other by the zipper sides 12.

A used state of the detachable heat-retaining glove according to the present invention will be described as follows. An assembling process of the detachable heat-retaining gloves of the present invention will not be described since it may be carried out in various modifications and embodiments according to purposes of those skilled in the art.

First, the heat-retaining member 4 and the outer skin member 6 are disposed at proper positions, and the body 2 of the tumbling doll shape which is foldable at the center is located.

Furthermore, the male zippers 8 are disposed from both side edges of the center of the body 2 to both side edges of the upper portion of the body 2.

After that, the female zippers 10 are disposed from both side edges of the center of the body 2 to both side edges of the lower portion of the body 2.

Moreover, the zipper slides 12 are disposed between the male zippers 8 and the female zippers 10 disposed at both sides of the body 2.

Here, in place of the male zipper 8, the female zipper 10 and the zipper slide 12, buttons, Velcro tapes or magnets may be used.

In addition, the receiving portion 14 is formed when the male zippers 8 and the female zippers 10 are engaged with each other by the zipper slides 12.

Additionally, the cross-shaped fitting portion 16 or the straight-shaped fitting portion 16a is formed at one side of the lower portion of the body 2.

Furthermore, the separation-preventing ring 18 is formed on one side of the inner face of the body 2.

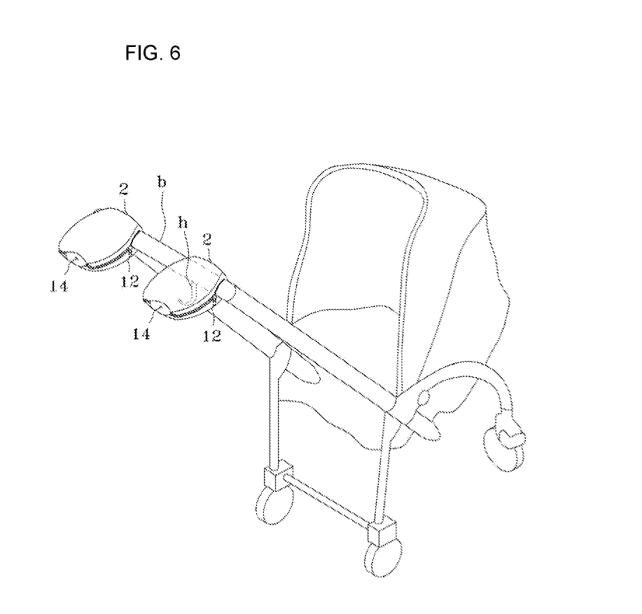

When the assembly is finished, the detachable heat-retaining gloves are installed on a baby carriage as shown in FIGS. 5 and 6.

A bar of the baby carriage is generally divided into a perpendicular bar (a) and a horizontal bar (b). The baby carriage having the horizontal bar (b) includes a hook (h) disposed at a side portion of the bar for allowing to hang articles, such as handbags, milk bags, caps or mobile phones, up on the hook.

In case of the baby carriage having the perpendicular bar (a), after the cross-shaped or straight-shaped fitting portion 16 or 16a of the body 2 is fit onto the perpendicular bar (a), the separation-preventing ring 18 mounted at one side of the inner face of the body 2 is hung on the perpendicular bar (a) to prevent a movement of the body 2, and then, the male zippers 8 and the female zippers 10 disposed at both sides of the body 2 are engaged with each other by the zipper slips 12. Then, the detachable heat-retaining gloves according to the present invention are ready to be used.

In the meantime, in case of the baby carriage having the horizontal bar (b), after the cross-shaped or straight-shaped fitting portion 16 or 16a of the body 2 is fit onto the hook (h) of the horizontal bar (b), a central portion of the body 2 which is foldable at the central portion, becomes in close contact with the horizontal bar (b), and then, the male zippers 8 and the female zippers 10 disposed at both sides of the body 2 are engaged with each other by the zipper slips 12. Then, the detachable heat-retaining gloves according to the present invention are ready to be used.

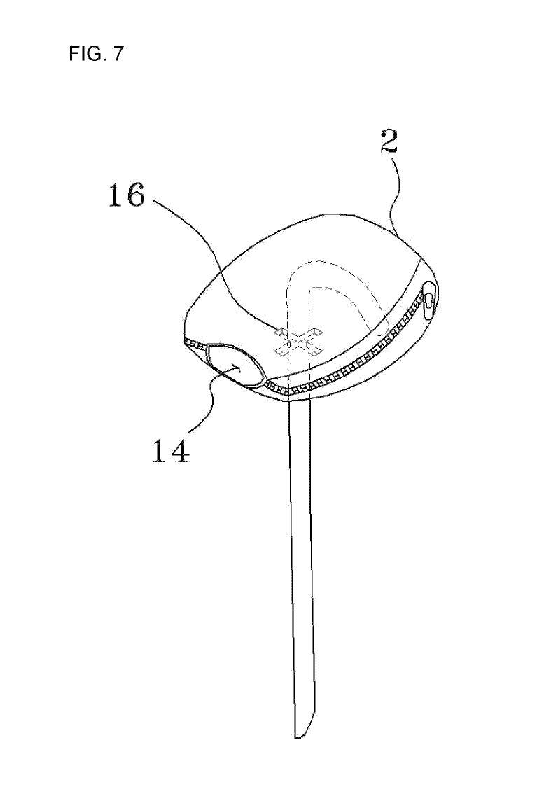

Meanwhile, as shown in FIG. 7, the detachable heat-retaining gloves according to the present invention can be applied to a stick.

As described above, it should be understood that the embodiments described herein are described as exemplifications for explaining the present invention and do not restrict the meanings and scope of the present invention. Thus, it is to be appreciated to those skilled in the art that the present invention is meant to cover all modifications, similarities and alternatives which are included in the spirit and scope of the present invention.

Also, it should be understood that the technically protective scope of the present invention is defined by the idea of claims of the present invention.

What is claimed is:

1. Detachable heat-retaining gloves, each of which includes:
a body of a tumbling doll shape symmetrically formed and being foldable at a central portion thereof;
a heat-retaining member joined along the inner face of the body;
an outer skin member joined along the outer face of the body;
first and second zippers joined along both side edges of the body to form a receiving portion inside the body; and
a separation-preventing ring mounted at one side of the inner face of the body,
wherein the body further comprises a cross-shaped (+) or straight-shaped (−) fitting portion formed on one side of a lower portion of the body.

2. The detachable heat-retaining gloves according to claim 1, wherein buttons, Velcro tapes or magnets are capable of being mounted on the body in place of the zippers.

3. The detachable heat-retaining gloves according to claim 1, wherein heating wires are interposed between the heat-retaining member and the outer skin member.

4. The detachable heat-retaining gloves according to claim 1, further comprising wrist tightening portions extending from an upper end portion and a lower end portion of the body and formed symmetrically to form an entrance of the receiving portion.

5. The detachable heat-retaining gloves according to claim 1, further comprising a Velcro fastener mounted at an end portion of the fitting portion in order to prevent wind blowing into the receiving portion.
